(19) United States
(12) Patent Application Publication
(10) Pub. No.: US 2015/0126951 A1
(43) Pub. Date: May 7, 2015

(54) ADULT MALE URINARY INCONTINENCE ARTICLE

(71) Applicant: MARTIN MICHAEL SHARKEY, SAN ANTONIO, TX (US)

(72) Inventor: MARTIN MICHAEL SHARKEY, SAN ANTONIO, TX (US)

(21) Appl. No.: 14/450,057

(22) Filed: Aug. 1, 2014

Related U.S. Application Data
(60) Provisional application No. 61/962,283, filed on Nov. 5, 2013.

(51) Int. Cl. A61F 13/471; A61F 13/56; A61F 13/80; A61F 13/62

(52) U.S. Cl. CPC A61F 13/471; A61F 13/622; A61F 13/56; A61F 13/80; A61F 5/453
USPC 604/385.03

(57) ABSTRACT

The adult male urinary incontinence device is a disposable unit to catch urine from an adult male patient. The T-shaped device is comprised of a top layer that is fluid permeable, a middle layer that absorbs fluid, and a bottom layer that is fluid impermeable. The “T” shaped device is positioned under a patient's scrotum and rests on the upper thigh of the patient. The front flap of the device is folded over the patient's penis with the opposing side flaps folding over the front flap and fastened. The adult male urinary incontinence device allows the patient to be changed without moving the patient or awaking them.

### CROSS-REFERENCES TO RELATED APPLICATIONS

This application claims the benefit of U.S. provisional application Ser. No. 61/962,283 filed Nov. 5, 2013 entitled Adult Male Urinary Incontinence Article which is incorporated by reference herein.

### STATEMENT REGARDING FEDERALLY SPONSORED RESEARCH OR DEVELOPMENT

Not applicable.

### BACKGROUND OF THE INVENTION

1. Field of the Invention

The present invention relates to the field of adult incontinence pads/diapers.

2. Description of the Related Art

There are several products and methods to accommodate adult males suffering from urinary incontinence. The most common devices include the use of hour glass shaped diapers, indwelling urinary catheters, and suprapubic catheters. The catheters are invasive and have a high rate of infection. This also requires a tube to be continually running out of the patient. The current hourglass diapers also create complications as they are large and extend under the male patient's buttocks. To change a bedridden patient, a provider must role the patient onto his side to position the diaper and then role the patient to the other side to correctly position the diaper. This process is time consuming and physically demanding for the provider and patient alike. Many male patients suffering from urinary incontinence are physically weak and unable to assist the provider with rolling over. Furthermore, it is disruptive to a patient's sleep and creates unnecessary anxiety in the patient in dealing with routine changing. As a result, many healthcare providers delay changing male patients causing patients to suffer through soiled diapers which leads to skin break down and pressure ulcers.

### SUMMARY OF THE INVENTION

The adult male urinary incontinence device is a disposable unit to catch urine from an adult male patient. The device is comprised of a top layer that is fluid permeable, a middle layer that absorbs fluid, and a bottom layer that is fluid impermeable. The top layer and bottom layer are connected around their edges with the middle layer disposed between. The edges are sealed to prevent leakage of fluid. The device is “T” shaped with a front flap and two opposing side flaps. The top layer is positioned under the male patient's penis and scrotum with the back edge against the patient and the front flap extending away from the patient. The back edge contains elastic which allows for a more secured fit against the patient's scrotum without being overly tight or restrictive. In this configuration, the penis is positioned along the outer face of the center portion. The front flap is folded over the patient's penis. The opposing side flap is folded over the front flap. The other side flap is then folded over the first side flap such that a fastener may engage fastener receiving strip on the second side flap. As the patient urinates, the urine passes through the top layer and into the interior layer where the urine is stored. The bottom layer prevents the urine from leaking out and irritating the patient's skin. The adult male urinary incontinence device allows the patient to be changed without moving the patient or awaking the patient.

### BRIEF DESCRIPTION OF THE SEVERAL VIEWS OF THE DRAWINGS

FIG. 1 is a top view of an embodiment.

FIG. 2 is a bottom view of an embodiment.

FIG. 3 is a cross-section view of an embodiment.

FIG. 4 is a view of an embodiment positioned on a patient.

### DETAILED DESCRIPTION OF THE INVENTION

Referring to FIGS. 1, 2, and 3, the adult male urinary incontinence device 1 is comprised of a bottom layer 10, a top layer 30 and interior layer 50. The adult male urinary incontinence device 1 resembles a block “T” having a center portion 2, front flap portion 3, and opposing side flap portions 4, 5. The opposing side flap portions 4, 5 are positioned at the rear of the adult male urinary incontinence device 1 such that the back edge 6 is comprised of an edge of each opposing side flap portion 4, 5 and an edge of the center portion 2. The front flap portion 3 extends away from the back edge 6 and is positioned at an approximate ninety degree angle relative to each opposing side flap portion 4, 5.

Referring to FIG. 1, the bottom layer 10 resembles a block “T” having a center portion 12 with a front flap portion 14 and two opposing side flap portions 18, 22 extending therefrom. The front flap portion 14 has a front edge 15 and rounded side edges 16, 17. Each opposing side flap portion 18, 22 has a back edge 19, 23, opposing edge 20, 24, and a rounded front edge 21, 25. The back edge 11 of the bottom layer 10 is comprised of the back edges 19, 23 of each opposing side flap portions 18, 22 and back edge 13 of the center portion 12. The front flap portion 14 extends away from the center portion 12 and is positioned at an approximate ninety degree angle relative to each opposing side flap portions 18, 22. The rounded side edges 16, 17 of front flap portion 14 connect with respective rounded edges 21, 25 of each respective opposing side flap portions 18, 22.

The bottom layer 10 is constructed of a flexible nonwoven fabric sheet that forms the body of the diaper and is impervious to fluid. Nonwoven fabrics are made from plastic resins, such as nylon, polyester, polyethylene, or polypropylene. Polyethylene is the resin of choice for the non-permeable bottom layer as it is substantially impervious to fluid. Alternatively, the bottom layer 10 may be a woven material laminated with polyethylene such that polyethylene side faces the inner face of the top layer 30. In the disclosed embodiment, the bottom layer 10 is a single piece of material with all edges 13, 15, 16, 17, 19, 20, 21, 23, 24, 25 connect to form a continuous edge. In the disclosed embodiment the opposing side flap portions 18, 22 are identical in size. In the disclosed embodiment the front edge 15 of front flap portion 14 is longer than the opposing edge 20, 24 of each opposing side flap portion 18, 22. A wetness indicator may be positioned on the outside face 26 to show if the interior layer is wet.

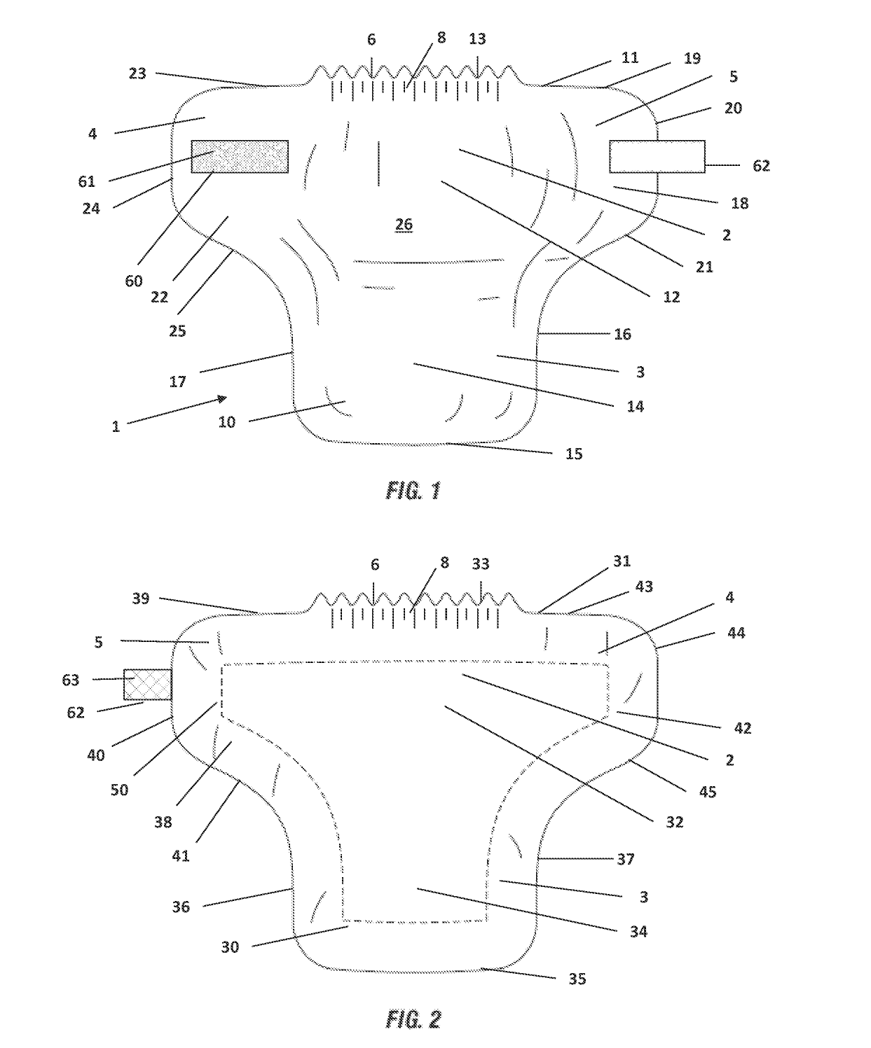

Referring to FIG. 2, the top layer 30 resembles a block “T” having a center portion 32 with a front flap portion 34 and two opposing side flap portions 38, 42 extending therefrom. The front flap portion 34 has a front edge 35 and rounded side edges 36, 37. Each opposing side flap portion 38, 42 has a back edge 39, 43, opposing edge 40, 44, and a rounded front edge 41, 45. The back edge 31 of the top layer 30 is comprised of the back edges 39, 43 of each opposing side flap portions 38, 42 and back edge 33 of the center portion 32. The front flap portion 34 extends away from the center portion 32 and is positioned at an approximate ninety degree angle relative to each opposing side flap portions 38, 42. The rounded side edges 36, 37 of front flap portion 34 connect with respective rounded edges 41, 45 of each respective opposing side flap portions 38, 42.

The top layer 30 is constructed of a flexible material that is fluid permeable such as a nonwoven fabric made from polypropylene. Polypropylene is typically the material used for the permeable top sheet. In the disclosed embodiment, the top layer 30 is a single piece of material with all edges 33, 35, 36, 37, 39, 40, 41, 43, 44, 45 connect to form a continuous edge. In the disclosed embodiment the opposing side flap portions 38, 42 are identical in size. In the disclosed embodiment the front edge 35 of front flap portion 34 is longer than the opposing edge 40, 44 of each opposing side flap portion 38, 42.

In the disclosed embodiment, the bottom layer 10 and top layer 30 are substantially identical in dimensions such that center portion 12, front flap portion 14, and opposing side flap portions 18, 22 of the bottom layer 10 correspond and match in shape the center portion 32, front flap portion 34, and opposing side flap portions 38, 42 of the top layer 30.

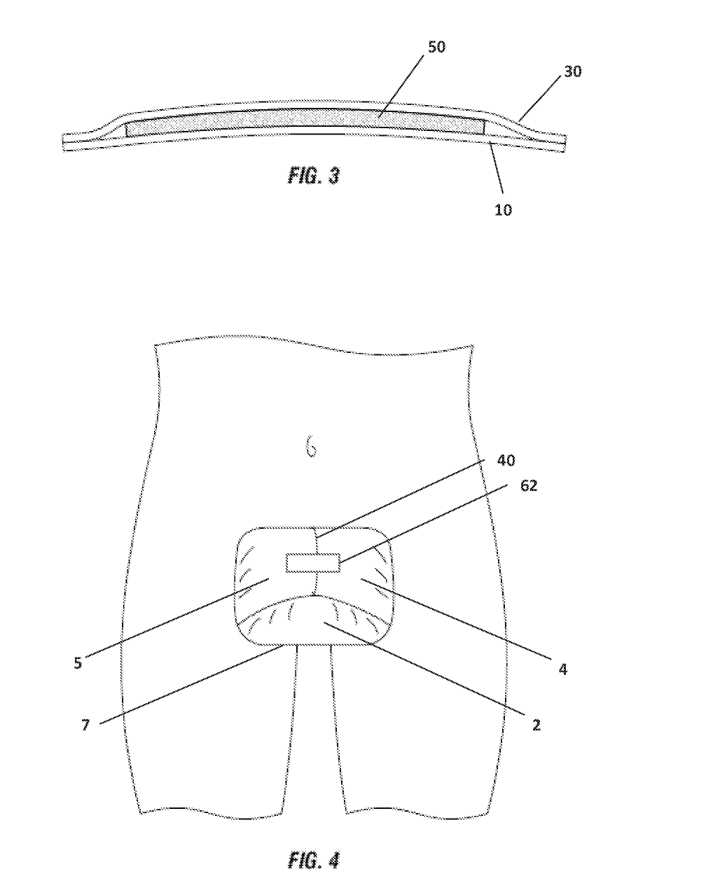

Referring to FIGS. 2 and 3, interior layer 50 is disposed between the bottom layer 10 and top layer 30. The interior layer 50 resembles a block “T,” similar to the bottom layer 10 and top layer 30. The interior layer 50 is made of a flexible material that is fluid absorbent. This layer may be composed of two elements, a hydrophilic polymer and a fibrous material such as wood pulp. The polymer is made of fine particles of an acrylic acid derivative, such as sodium acrylate, potassium acrylate, or an alkyl acrylate. Polymers with this ability are referred to as hydrogels, superabsorbents, or hydrocolloids.

In the disclosed embodiment the top layer 30 and bottom layer 10 are bonded together with the interior layer 50 disposed between. Such bonding may occur by mechanically, chemically, or thermally interlocking the plastic fibers. The three components are in long strips and must be joined together and cut into diaper-sized units. This is accomplished by feeding the absorbent pad onto a conveyor with the polyethylene bottom sheet. The polypropylene top sheet is then fed into place, and the compiled sheets are joined by gluing, heating, or ultrasonic welding. The bonding occurs along bottom layer edges 13, 15, 16, 17, 19, 20, 21, 23, 24, 25 and the respective counterpart top layer edges 33, 35, 36, 37, 39, 40, 41, 43, 44, 45. The bonding or sealing prevents any leakage of fluid out of the sides.

In the disclosed embodiment, elastic fibers are attached to the sheets through the back edge 11 of the bottom layer 10 and the back edge 31 of the top layer 30. After the diaper is assembled the elastic fibers cause the back edges 11, 31 to gather or bunch the edges of the diaper into the proper shape so it fits snugly around the user's crotch. It creates a curvature of the back edge 6 which pulls opposing side flap portions 4, 5 away from the front flap portion 3. Alternatively, an elastic band may be stitched or adhered between the top layer 30 and bottom layer 10.

Disposed on the outer face 26 of the opposing side flap portion 22 is a fastener receiving strip 60. In the disclosed embodiment the fastener receiving strip 60 is rectangular in shape with the length dimension extending from the opposing edge 24 towards the center portion 12. The fastener receiving strip 60 contains a fastening receiving material 61 on its exposed face. On the other opposing side flap portion 18 a fastener 62 is attached to the outer face 26. The fastener 62 is partially attached with a portion of the fastener 62 extending beyond the opposing edge 20. As seen in FIG. 2, the interior face of fastener 62 contains a fastening material 63. In the disclosed embodiment the fastening material 63 and fastening receiving material 61 are hook and loop fasteners. Other fastening material and fastening receiving material are suitable such as weak adhesives.

Use of the Adult Male Urinary Incontinence Device 1 is described in relation to FIG. 4. The top layer 30 is positioned under the male patient's penis and scrotum with the back edge 6 against the patient and the front edge 7 extending away from the patient. The device 1 rests on top of the patient's upper thighs. The back edge 6, with the elastic, allows for a more secured fit against the patient's scrotum without being overly tight or restrictive. In this configuration, the penis is positioned along the outer face of the center portion 32. The front flap 2 is folded over the patient's penis. The opposing side flap portion 4 is folded over the front flap portion 3. The opposing side flap portion 5 is then folded over the opposing side flap portion 4 such that fastener 62 may engage fastener receiving strip 60 on opposing side flap portion 4. As the patient urinates, the urine passes through the top layer 30 and into the interior layer 50 where the urine is stored. The bottom layer 10 prevents the urine from leaking out and irritating the patient's skin.

The present invention is described above in terms of a preferred illustrative embodiment of an adult male urinary incontinence device. Those skilled in the art will recognize that alternative constructions of such a device can be used in carrying out the present invention. Aspects such as the material used, fasteners, and dimensions are changeable without affecting the principles of the invention. Other aspects, features, and advantages of the present invention may be obtained from a study of this disclosure and the drawings, along with the appended claims.

I claim:

1. A urinary incontinence device comprising:
a bottom layer having a center portion, a front flap portion extending from said center portion, a first side flap extending from said center portion, and a second side flap extending from said center portion wherein said second side flap opposes said first side flap;
a top layer having a center portion, a front flap portion extending from said center portion, a first side flap extending from said center portion, and a second side flap extending from said center portion wherein said second side flap opposes said first side flap;
an interior layer positioned in between said bottom layer and said top layer; and
said bottom layer is secured to said top layer such that said center portion of said bottom layer is aligned with said center portion of said top layer, said front flap portion of said bottom layer is aligned with said front flap of said top layer, said first side flap portion of said bottom layer is aligned with said first side flap of said top layer, and said second side flap portion of said bottom layer is aligned with said second side flap of said top layer.

2. The urinary incontinence device of claim 1 wherein said front flap of said bottom layer is positioned perpendicular to said first side flap and said second side flap of said bottom layer.

3. The urinary incontinence device of claim 2 wherein said bottom layer and said top layer are predominately “T” shaped.

4. The urinary incontinence device of claim 3 wherein said first side flap and said second side flap are equal in size.

5. The urinary incontinence device of claim 4 wherein said front flap of said bottom layer is longer than said first side flap of said bottom layer.

6. The urinary incontinence device of claim 5 wherein said top layer is substantially coextensive with said bottom layer.

7. The urinary incontinence device of claim 3 wherein said interior layer is predominately “T” shaped.

8. The urinary incontinence device of claim 1 wherein said bottom layer is constructed of a flexible material that is fluid impermeable.

9. The urinary incontinence device of claim 1 wherein said top layer is constructed of a flexible material that is fluid permeable.

10. The urinary incontinence device of claim 1 wherein said interior layer is constructed of a flexible material that is fluid absorbent.

11. The urinary incontinence device of claim 1 further comprising a fastener partially attached on said first side flap of said bottom layer and a fastener receiving strip on said second side flap of said bottom layer.

12. The incontinence device of claim 11 wherein said fastener and said fastener receiving strip are hook and loop fasteners.

13. A urinary incontinence device comprising:
an elongate body with a first side flap and a second side flap opposing said first side flap wherein said elongate body, said first side flap and said second side flap are comprised of a bottom layer joined to a top layer; and
an interior layer positioned in between said bottom layer and said top layer.

14. A urinary incontinence device of claim 13 wherein said first side flap and said second side flap are positioned at a first end of said elongate body.

15. A urinary incontinence device of claim 13 wherein said first end contains an elastic band positioned between said top layer and said bottom layer.

16. The urinary incontinence device of claim 13 wherein said bottom layer is constructed of a flexible material that is fluid impermeable.

17. The urinary incontinence device of claim 13 wherein said top layer is constructed of a flexible material that is fluid permeable.

18. The urinary incontinence device of claim 13 wherein said interior layer is constructed of a flexible material that is fluid absorbent.

19. The urinary incontinence device of claim 18 further comprising a fastener partially attached on said first side flap of said bottom layer and a fastener receiving strip on said second side flap of said bottom layer.

20. The incontinence device of claim 19 wherein said fastener and said fastener receiving strip are hook and loop fasteners.
